SEQUENCE LISTING (11 sequences)

SEQ ID NO: 1 - protein, 122 aa, Homo sapien
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 2 - protein, 107 aa, Homo sapien
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 3 - protein, 5 aa, Homo sapien
DYAMH

SEQ ID NO: 4 - protein, 17 aa, Homo sapien
TISWNSGSIGYADSVKG

SEQ ID NO: 5 - protein, 13 aa, Homo sapien
DIQYGNYYYGMDV

SEQ ID NO: 6 - protein, 11 aa, Homo sapien
RASQSVSSYLA

SEQ ID NO: 7 - protein, 7 aa, Homo sapien
DASNRAT

SEQ ID NO: 8 - protein, 9 aa, Homo sapien
QQRSNWPIT

SEQ ID NO: 9 - protein, 17 aa, Homo sapien
DYYGAGSFYDGLYGMDV

SEQ ID NO: 10 - protein, 81 aa, Homo sapien
DYAMHWVRQAPGKGLEWVSTISWNSGSIGYADSVKGRFTISRDNAKKSLYLQMNSLRAEDTALYYCAKDIQYGNYYYGMDV

SEQ ID NO: 11 - protein, 14 aa, Homo sapien
DNQYGSGSTYGLGV

(19) United States
(12) Patent Application Publication
(10) Pub. No.: US 2011/0274697 A1
(43) Pub. Date: Nov. 10, 2011

(54) NOVEL USES

(76) Inventors: Cherry Teresa Thomas, King of Prussia, PA (US); Geoffrey Chan, King of Prussia, PA (US)

(21) Appl. No.: 13/144,335

(22) PCT Filed: Jan. 15, 2010
(86) PCT No.: PCT/US10/21123

§ 371 Date: Jul. 13, 2011

Related U.S. Application Data
(60) Provisional application No. 61/145,210, filed on Jan. 16, 2009.

(51) Int. Cl. A61K 39/395; A61P 35/00

(52) U.S. Cl. 424/142.1; 424/173.1

(57) ABSTRACT

The present invention relates generally to the use of bendamustine in combination with an anti-CD20 antibody to treat cancer.

### CROSS REFERENCE TO RELATED APPLICATION

This application claims priority to U.S. patent application Ser. No. 61/145,210 filed Jan. 16, 2009, which is incorporated by reference in its entirety.

### FIELD OF INVENTION

The present invention relates to the use of bendamustine in combination with an anti-CD20 antibody to treat cancer.

### BACKGROUND OF THE INVENTION

Indolent Non-Hodgkin's Lymphomas (IL) are slow growing forms of lymphoma. They encompass what were called low grade and some categories of intermediate grade NHL in the Working Formulation. If patients are not cured in very early stage and low grade disease, the goal of treatment is palliative. FL is the second most common lymphoma in US and Europe, accounting for 11% to 35% of all Non Hodgkins Lymphoma (NHL) [WHO 2001]. Follicular lymphoma (FL) belongs to the group of indolent lymphomas and is a subgroup of mature (peripheral) B cell neoplasms [WHO 2001]. It is defined as a lymphoma of germinal center B cells (centrocytes and centroblasts) which have at least a partially follicular pattern.

Although indolent lymphoma is well-treated with rituximab-based therapies, options are limited with those who become refractory to rituximab. Bendamustine is a synthetic nitrogen mustard compound that has shown activity in the treatment of rituximab refractory indolent lymphoma and has been shown to have activity in subjects refractory to other alkylators. However, alternative therapies are especially required in subjects refractory to these early therapies.

Recently there has been many reports of new generation of anti-CD20 antibodies. One such novel antibody is ofatumumab. Ofatumumab is a new generation, human monoclonal antibody that targets a distinct membrane proximal, small loop epitope (specific binding site) of the CD20 molecule on the surface of B-cells. This generates a superior induction of tumor cell lysis by CDC (complement dependent cytotoxicity) activity, especially in cells with low CD20 density, as is the case in CLL, with similar ADCC (antibody-dependent cell mediated cytotoxicity) activity, compared to tumor cell lysis capability observed with rituximab. Ofatumumab described as 2F2 antibody in WO2004/035607 is in clinical development for the treatment of non-Hodgkin's lymphoma (NHL), chronic lymphocytic leukemia (CLL), and rheumatoid arthritis (RA). See also Teeling et al., Blood, 104, pp 1793 (2004); and Teeling et al., J. Immunology, 177, pp 362-371 (2007).

### SUMMARY OF THE INVENTION

In one embodiment, the present invention relates a method of treating to any cancer (tumor) expressing CD20, including, precursor B- and T-cell neoplasms, mature B-cell neoplasms, Hodgkin's lymphoma, and immunodeficiency associated lymphoproliferative disorders in a human patient, comprising the step of administering to the patient an anti-CD20 antibody in combination with bendamustine. In one embodiment the administration is simultaneous. In another embodiment the administration is sequential in which bendamustine is administered first. Yet in another embodiment an anti-CD20 antibody is administered first. In yet in another embodiment, administration of an anti-CD20 antibody and bendamustine is staggered.

In one embodiment the invention relates to a method of treating rituximab refractory indolent non-Hodgkin's Lymphoma, including FL (follicular lymphoma), in a human patient, comprising the step of administering to the patient an anti-CD20 antibody in combination with bendamustine. In one embodiment the administration is simultaneous. In another embodiment the administration is sequential in which bendamustine is administered first. Yet in another embodiment an anti-CD20 antibody is administered first. In yet in another embodiment, administration of an anti-CD20 antibody and bendamustine is staggered.

In one embodiment the invention relates to a pharmaceutical composition comprising bendamustine and an anti-CD20 antibody wherein the combination is suitable for separate, sequential and/or simultaneous administration.

In one embodiment the anti-CD20 antibody is an isolated human anti-CD20 antibody which binds to an epitope on CD20 which does not comprise or require the amino acid residue proline at position 172, but which comprises or requires the amino acid residues asparagine at position 163 and asparagine at position 166. Examples of such antibodies are found in WO2004/035607.

In another embodiment the anti-CD20 antibody is ofatumuamb.

In one embodiment the invention relates to the use of an anti-CD20 antibody (in particular ofatumumab) in the manufacture of a medicament for the treatment of cancer (in particular rituximab-refractory indolent non-Hodgkin's lymphoma), wherein the medicament is for administration in combination therapy with bendamustine.

In one embodiment, the invention relates to an anti-CD20 antibody (in particular ofatumuamb) for use in the treatment of cancer (in particular rituximab-refractory indolent non-Hodgkin's lymphoma) in combination with bendamustine.

### DESCRIPTION OF THE FIGURES

FIG. 1 depicts a non-limiting example of ofatumumab/bendamustine administration.

FIG. 2 depicts medium level expression profile of CD20 on JVM-3 cells. First peak: Mab control; second peak: BD Bioscience anti-CD20 antibody clone 2H7; third peak: rituxan; fourth peak: ofatumumab.

FIG. 3 depicts advantage of ofatumumab/bendamustine combination in suboptimal doses (ofatumumab: 2 mg/kg & bendamustine 50 mg/kg; n=6/group)

FIG. 4 depicts combination of ofatumumab and bendamustine (TREANDA) in JVM3 (CLL) model (s.c. day 24; n=6/group)

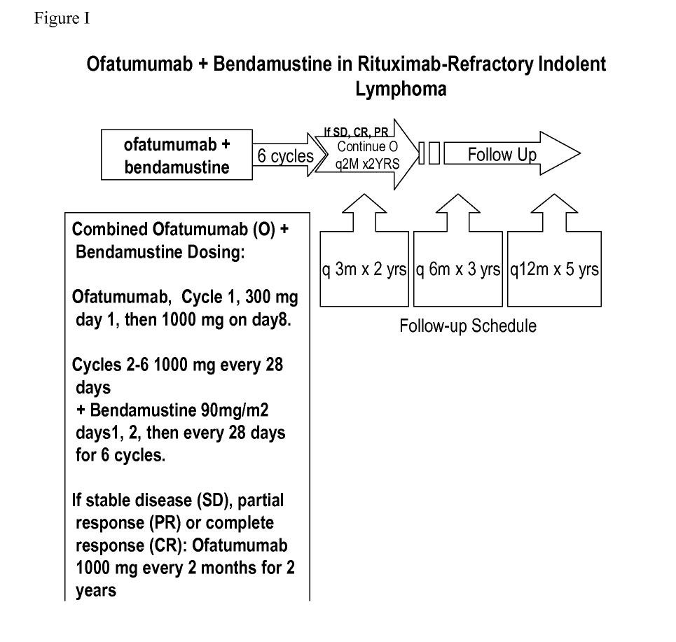

### DETAILED DESCRIPTION

Current treatment strategies for follicular lymphoma focus on establishing maximal disease control and prolonged survival. Disease that has advanced beyond early stage and low grade histology remain incurable. There is a balance between achieving effective therapy without toxicity. Therefore, there is still an unmet need for effective therapies with limited side effects for the treatment of the majority of FL subjects, especially those who become refractory to alkylators, purine analogues, and rituximab. Ofatumumab has shown activity in rituximab resistant subjects [Hagenbeek, et al. Blood 2008; 111:5486-5495] and bendamustine has shown activity in alkylator and purine analogue resistant subjects [Schoffski et al., Ann Oncol. 2000; 11:729-734; Solal-Celigny et al., Blood. 2004; 104:1258-1265; Heider et al., Anticancer Drugs. 2001; 12:725-729; Bremer K., J Cancer Res Clin Oncol 2002; 128:603-609; Friedberg et al., J Clin Oncol. 2008; 26:204-210]. Combination of ofatumumab and bendamustine combines efficacy with a low toxicity profile for subjects who become refractory to other treatment modalities.

The invention relates to a method of treating rituximab refractory indolent non-Hodgkin's Lymphoma, including FL (follicular lymphoma), in a human patient, comprising the step of administering to the patient an anti-CD20 antibody in combination with bendamustine. In one embodiment the administration is simultaneous. In another embodiment the administration is sequential in which bendamustine is administered first. Yet in another embodiment an anti-CD20 antibody is administered first. In yet in another embodiment, administration of an anti-CD20 antibody and bendamustine is staggered.

The invention also relates to a method of treating to a tumor type expressing CD20 in a human patient, comprising the step of administering to the patient an anti-CD20 antibody in combination with bendamustine. In one embodiment the administration is simultaneous. In another embodiment the administration is sequential in which bendamustine is administered first. Yet in another embodiment an anti-CD20 antibody is administered first. In yet in another embodiment, administration of an anti-CD20 antibody and bendamustine is staggered. An example of tumor type expressing CD20 include a group selected from a precursor B- or T-cell neoplasm, a mature B-cell neoplasm, Hodgkin's lymphoma, or an immunodeficiency associated lymphoproliferative disorder.

Non-limiting way to dose bendamustine and ofatumuamb is exemplified in Example 1.

The invention also relates to a method of treating a cancer selected from the group consisting of NHL (non-Hodgkin's lymphoma), B cell lymphoblastic leukemia/lymphoma, mature B cell neoplasms, B cell chronic lymhocytic leukemia (CLL), small lymphocytic lymphoma (SLL), B cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, anaplastic large-cell lymphoma (ALCL), T-cell Non-Hodgkin's lymphoma; and melanoma comprising administering to a human patient ofatumuamb and bendamustine. In one embodiment the administration is simultaneous. In another embodiment the administration is sequential in which bendamustine is administered first. Yet in another embodiment an anti-CD₂O antibody is administered first. In yet in another embodiment, administration of an anti-CD20 antibody and bendamustine is staggered.

Rituximab (R) refractory indolent lymphoma is defined as follows. Lymphoma is refractory to rituximab given as monotherapy or in combination with any chemotherapy or to rituximab given as maintenance treatment following rituximab plus chemotherapy. Lymphoma is refractory if there is:
1. Failure to achieve at least partial response (PR) to rituximab given as monotherapy or in combination with any chemotherapy; or,
2. Disease progression while on rituximab (either given as monotherapy or in combination with any chemotherapy or during rituximab maintenance treatment following R-chemo); or,
3. Disease progression in responders within 6 months of the last dose of rituximab (either given as monotherapy or in combination with any chemotherapy or after rituximab maintenance treatment schedule following R-chemo)

In one embodiment of the invention, the anti-CD20 antibody is monoclonal.

In one embodiment, the anti-CD20 antibody has Fc mediated effector function.

In one embodiment, the anti-CD20 antibody has antibody-dependent-cell-mediated cytoxicity (ADCC) effector function.

In one embodiment, the anti-CD20 antibody has complement-dependent-cytoxicity (CDC) effector function.

In one embodiment of the invention, the anti-CD20 antibody is a chimeric, humanized or human monoclonal antibody.

In one embodiment, the monoclonal antibody against CD20 (anti-CD₂O antibody) is a full-length antibody selected from the group consisting of a full-length IgG1 antibody, a full-length IgG2 antibody, a full-length IgG3 antibody, a full-length IgG4 antibody, a full-length IgM antibody, a full-length IgA1 antibody, a full-length IgA2 antibody, a full-length secretory IgA antibody, a full-length IgD antibody, and a full-length IgE antibody, wherein the antibody is glycosylated in a eukaryotic cell.

In one embodiment, the anti-CD20 antibody is a full-length antibody, such as a full-length IgG1 antibody.

In one embodiment, the anti-CD20 antibody is an antibody fragment, such as a scFv or a UniBody™ (a monovalent antibody as disclosed in WO 2007/059782). In one embodiment of the invention, the antibody against CD20 (anti-CD20 antibody) is a binding-domain immunoglobulin fusion protein comprising (i) a binding domain polypeptide in the form of a heavy chain variable region of SEQ ID NO:1 or a light chain variable region of SEQ ID NO:2 that is fused to an immunoglobulin hinge region polypeptide, (ii) an immunoglobulin heavy chain CH2 constant region fused to the hinge region, and (iii) an immunoglobulin heavy chain CH3 constant region fused to the CH2 constant region.

In one embodiment, the antibody against CD20 binds to mutant P172S CD20 (proline at position 172 mutated to serine) with at least the same affinity as to human CD20.

In one embodiment of the invention, the antibody against CD20 binds to an epitope on CD20

(i) which does not comprise or require the amino acid residue proline at position 172;

(ii) which does not comprise or require the amino acid residues alanine at position 170 or proline at position 172;

(iii) which comprises or requires the amino acid residues asparagine at position 163 and asparagine at position 166;

(iv) which does not comprise or require the amino acid residue proline at position 172, but which comprises or requires the amino acid residues asparagine at position 163 and asparagine at position 166; or

(v) which does not comprise or require the amino acid residues alanine at position 170 or proline at position 172, but which comprises or requires the amino acid residues asparagine at position 163 and asparagine at position 166.

In one embodiment, the antibody against CD20 binds to an epitope in the small first extracellular loop of human CD20.

In one embodiment, the antibody against CD20 binds to a discontinuous epitope on CD20.

In one embodiment, the antibody against CD20 binds to a discontinuous epitope on CD20, wherein the epitope comprises part of the first small extracellular loop and part of the second extracellular loop.

In one embodiment, the antibody against CD20 binds to a discontinuous epitope on CD20, wherein the epitope has residues AGIYAP of the small first extracellular loop and residues MESLNFIRAHTPYI of the second extracellular loop.

In one embodiment, the antibody against CD20 has one or more of the characteristics selected from the group consisting of:

(i) capable of inducing complement dependent cytotoxicity (CDC) of cells expressing CD20 in the presence of complement;

(ii) capable of inducing complement dependent cytotoxicity (CDC) of cells expressing CD20 and high levels of CD55 and/or CD59 in the presence of complement;

(iii) capable of inducing apoptosis of cells expressing CD20;

(iv) capable of inducing antibody dependent cellular cytotoxicity (ADCC) of cells expressing CD20 in the presence of effector cells;

(v) capable of inducing homotypic adhesion of cells which express CD20;

(vi) capable of translocating into lipid rafts upon binding to CD20;

(vii) capable of depleting cells expressing CD20;

(viii) capable of depleting cells expressing low levels of CD20 (CD20 low cells); and

(ix) capable of effectively depleting B cells in situ in human tissues.

In one embodiment of the invention, the antibody against CD20 comprises a VH CDR3 sequence selected from SEQ ID NOs: 5, 9, or 11.

In one embodiment, the antibody against CD20 comprises a VH CDR1 of SEQ ID NO:3, a VH CDR2 of SEQ ID NO:4, a VH CDR3 of SEQ ID NO:5, a VL CDR1 of SEQ ID NO:6, a VL CDR2 of SEQ ID NO:7 and a VL CDR3 sequence of SEQ ID NO:8.

In one embodiment of the invention, the antibody against CD20 comprises a VH CDR1-CDR3 spanning sequence of SEQ ID NO:10.

In one embodiment of the invention, the antibody against CD20 has human heavy chain and human light chain variable regions comprising the amino acid sequences as set forth in SEQ ID NO:1 and SEQ ID NO:2, respectively; or amino acid sequences which are at least 95% identical, and more preferably at least 98%, or at least 99% identical to the amino acid sequences as set forth in SEQ ID NO:1 and SEQ ID NO:2, respectively.

In one embodiment of the invention an anti-CD20 antibody is selected from one of the anti-CD20 antibodies disclosed in WO 2004/035607, such as ofatumumab (2F2), 11B8, or 7D8, one of the antibodies disclosed in WO 2005/103081, such as 2C6, one of the antibodies disclosed in WO 2004/103404, AME-133 (humanized and optimized anti-CD20 monoclonal antibody, developed by Applied Molecular Evolution), one of the antibodies disclosed in US 2003/0118592, TRU-015 (CytoxB20G, a small modular immunopharma-ceutical fusion protein derived from key domains on an anti-CD20 antibody, developed by Trubion Pharmaceuticals Inc), one of the antibodies disclosed in WO 2003/68821, IMMU-106 (a humanized anti-CD20 monoclonal antibody), one of the antibodies disclosed in WO 2004/56312, ocrelizumab (2H7.v16, PRO-70769, R-1594), Bexxar® (tositumomab), and Rituxan®/MabThera® (rituximab). The terms “CD20” and “CD20 antigen” are used interchangeably herein, and include any variants, isoforms and species homologs of human CD20, which are naturally expressed by cells or are expressed on cells transfected with the CD20 gene. Synonyms of CD20, as recognized in the art, include B-lymphocyte surface antigen B1, Leu-16 and Bp35. Human CD20 has UniProtKB/Swiss-Prot entry P11836.

The term “immunoglobulin” as used herein refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH) and a heavy chain constant region. The heavy chain constant region, CH, typically is comprised of three domains, CH1, CH2, and CH3. Each light chain typically is comprised of a light chain variable region (abbreviated herein as VL) and a light chain constant region. The light chain constant region typically is comprised of one domain, CL. The VH and VL regions may be further subdivided into regions of hypervariability (or hypervariable regions which may be hypervariable in sequence and/or form of structurally defined loops), also termed complementarity determining regions

(CDRs), interspersed with regions that are more conserved, termed framework regions

(FRs).

Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases, such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (for instance residue 52a according to Kabat) after residue 52 of VH CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “antibody” as used herein refers to an immunoglobulin molecule, a fragment of an immunoglobulin molecule, or a derivative of either thereof, which has the ability to specifically bind to an antigen under typical physiological conditions for a significant period of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or a time sufficient for the antibody to recruit an Fc-mediated effector activity).

The variable regions of the heavy and light chains of the immunoglobulin molecule contain a binding domain that interacts with an antigen. The constant regions of the antibodies may mediate the binding of the immunoglobulin to host tissues or factors, including various cells of the immune system (such as effector cells) and components of the complement system such as C1q, the first component in the classical pathway of complement activation.

The anti-CD20 antibody may be mono-, bi- or multispecific. Indeed, bispecific antibodies, diabodies, and the like, provided by the present invention may bind any suitable target in addition to a portion of CD20.

As indicated above, the term “antibody” as used herein, unless otherwise stated or clearly contradicted by the context, includes fragments of an antibody provided by any known technique, such as enzymatic cleavage, peptide synthesis and recombinant techniques that retain the ability to specifically bind to an antigen. It has been shown that the antigen-binding function of an antibody may be performed by fragments of a full-length (intact) antibody. Examples of antigen-binding fragments encompassed within the term “antibody” include, but are not limited to (i) a Fab fragment, a monovalent fragment consisting of the VL, VH, CL and CH1 domains; (ii) F(ab)2 and F(ab′)2 fragments, bivalent fragments comprising two Fab fragments linked by a disulfide bridge at the hinge region; (iii) a Fd fragment consisting essentially of the VH and CH1 domains; (iv) a Fv fragment consisting essentially of the VL and VH domains of a single arm of an antibody, (v) a dAb fragment (Ward et al., Nature 341, 544-546 (1989)), which consists essentially of a VH domain and also called domain antibodies (Holt et al. (November 2003) Trends Biotechnol. 21(11):484-90); (vi) a camelid antibody or nanobody (Revets et al. (January 2005) Expert Opin Biol Ther. 5(1):111-24), (vii) an isolated complementarity determining region (CDR), such as a VH CDR3, (viii) a UniBody™, a monovalent antibody as disclosed in WO 2007/059782, (ix) a single chain antibody or single chain Fv (scFv), see for instance Bird et al., Science 242, 423-426 (1988) and Huston et al., PNAS USA 85, 5879-5883 (1988)), (x) a diabody (a scFv dimer), which can be monospecific or bispecific (see for instance PNAS USA 90(14), 6444-6448 (1993), EP 404097 or WO 93/11161 for a description of diabodies), a triabody or a tetrabody. Although such fragments are generally included within the definition of an antibody, they collectively and each independently are unique features of the present invention, exhibiting different biological properties and utility. These and other useful antibody fragments in the context of the present invention are discussed further herein.

It should be understood that the term antibody generally includes monoclonal antibodies as well as polyclonal antibodies. The antibodies can be human, humanized, chimeric, murine, etc. An antibody as generated can possess any isotype.

The term “human antibody”, as used herein, is intended to include antibodies having variable and constant regions derived from human germline immunoglobulin sequences. The human antibodies of the present invention may include amino acid residues not encoded by human germline immunoglobulin sequences (for instance mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted into human framework sequences.

As used herein, a human antibody is “derived from” a particular germline sequence if the antibody is obtained from a system using human immunoglobulin sequences, for instance by immunizing a transgenic mouse carrying human immunoglobulin genes or by screening a human immunoglobulin gene library, and wherein the selected human antibody is at least 90%, such as at least 95%, for instance at least 96%, such as at least 97%, for instance at least 98%, or such as at least 99% identical in amino acid sequence to the amino acid sequence encoded by the germline immunoglobulin gene. Typically, a human antibody derived from a particular human germline sequence will display no more than 10 amino acid differences, such as no more than 5, for instance no more than 4, 3, 2, or 1 amino acid difference from the amino acid sequence encoded by the germline immunoglobulin gene. For VH antibody sequences the VH CDR3 domain is not included in such comparison.

The term “chimeric antibody” refers to an antibody that contains one or more regions from one antibody and one or more regions from one or more other antibodies. The term “chimeric antibody” includes monovalent, divalent, or polyvalent antibodies. A monovalent chimeric antibody is a dimer (HL)) formed by a chimeric H chain associated through disulfide bridges with a chimeric L chain. A divalent chimeric antibody is a tetramer (H2L2) formed by two HL dimers associated through at least one disulfide bridge. A polyvalent chimeric antibody may also be produced, for example, by employing a CH region that assembles into a molecule with 2+binding sites (for instance from an IgM H chain, or u chain). Typically, a chimeric antibody refers to an antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see for instance U.S. Pat. No. 4,816,567 and Morrison et al., PNAS USA 81, 6851-6855 (1984)). Chimeric antibodies are produced by recombinant processes well known in the art (see for instance Cabilly et al., PNAS USA 81, 3273-3277 (1984), Morrison et al., PNAS USA 81, 6851-6855 (1984), Boulianne et al., Nature 312, 643-646 (1984), EP125023, Neuberger et al., Nature 314, 268-270 (1985), EP171496, EP173494, WO 86/01533, EP184187, Sahagan et al., J. Immunol. 137, 1066-1074 (1986), WO 87/02671, Liu et al., PNAS USA 84, 3439-3443 (1987), Sun et al., PNAS USA 84, 214-218 (1987), Better et al., Science 240, 1041-1043 (1988) and Harlow et al., Antibodies: A Laboratory Manual, Cold Spring Harbor Laboratory Press, Cold Spring Harbor, N.Y., (1988)).

The term “humanized antibody” refers to a human antibody which contain minimal sequences derived from a non-human antibody. Typically, humanized antibodies are human immunoglobulins (recipient antibody) in which residues from a hypervariable region of the recipient are replaced by residues from a hypervariable region of a non-human species (donor antibody), such as mouse, rat, rabbit or non-human primate having the desired specificity, affinity, and capacity.

Furthermore, humanized antibodies may comprise residues which are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. A humanized antibody optionally also will comprise at least a portion of a human immunoglobulin constant region. For further details, see Jones et al., Nature 321, 522-525 (1986), Riechmann et al., Nature 332, 323-329 (1988) and Presta, Curr. Op. Struct. Biol. 2, 593-596 (1992).

The term “patient” refers to a human patient.

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal nonhuman animal, such as a transgenic mouse, having a genome comprising a human heavy chain transgene and a light chain transgene, fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further elsewhere herein), (b) antibodies isolated from a host cell transformed to express the antibody, such as from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The terms “transgenic, non-human animal” refers to a non-human animal having a genome comprising one or more human heavy and/or light chain transgenes or transchromosomes (either integrated or non-integrated into the animal's natural genomic DNA) and which is capable of expressing fully human antibodies. For example, a transgenic mouse can have a human light chain transgene and either a human heavy chain transgene or human heavy chain transchromosome, such that the mouse produces human anti-CD20 antibodies when immunized with CD20 antigen and/or cells expressing CD20. The human heavy chain transgene may be integrated into the chromosomal DNA of the mouse, as is the case for transgenic mice, for instance the HuMAb-Mouse®, such as HCo7 or HCo12 mice, or the human heavy chain transgene may be maintained extrachromosomally, as is the case for the transchromosomal KM-Mouse® as described in WO 02/43478. Such transgenic and transchromosomal mice (collectively referred to herein as “transgenic mice”) are capable of producing multiple isotypes of human monoclonal antibodies to a given antigen (such as IgG, IgA, IgM, IgD and/or IgE) by undergoing V-D-J recombination and isotype switching. Transgenic, nonhuman animals can also be used for production of antibodies against a specific antigen by introducing genes encoding such specific antibody, for example by operatively linking the genes to a gene which is expressed in the milk of the animal.

For amino acid (polypeptide) sequences, the term “identity” or “homology” indicates the degree of identity between two amino acid sequences when optimally aligned and compared with appropriate insertions or deletions. The percent identity between two sequences is a function of the number of identical positions shared by the sequences (i.e., % identity=number of identical positions/total number of positions times 100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two polypeptide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

By way of example, a polypeptide sequence may be identical to a polypeptide reference sequence as described herein (for example SEQ ID NO: 1) that is be 100% identical, or it may include up to a certain integer number of amino acid alterations as compared to the reference sequence such that the % identity is less than 100%, such as at least 50, 60, 70, 75, 80, 85, 90, 95, 98, or 99% identical. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by the polypeptide reference sequence as described herein (for example SEQ ID NO: 1) by the numerical percent of the respective percent identity (divided by 100) and then subtracting that product from said total number of amino acids in the polypeptide reference sequence as described herein (for example SEQ ID NO: 1), or:

nₐ≦xₐ−(xₐ·y),

wherein nₐ is the number of amino acid alterations, xₐ is the total number of amino acids in the polypeptide sequence encoded by SEQ ID NO: 1, and y is, 0.50 for 50%, 0.60 for 60%, 0.70 for 70%, 0.75 for 75%, 0.80 for 80%, 0.85 for 85%, 0.90 for 90%, 0.95 for 95%, 0.98 for 98%, 0.99 for 99%, or 1.00 for 100%, · is the symbol for the multiplication operator, and wherein any non-integer product of xₐ and y is rounded down to the nearest integer prior to subtracting it from xₐ.

The present invention also provides pharmaceutical compositions (formulations) comprising bendamustine. Such compositions comprise a therapeutically effective amount of bendamustine, and may further comprise a pharmaceutically acceptable carrier, diluent, or excipient. Such pharmaceutical carriers can be sterile liquids, such as water and oils, including those of petroleum, animal, vegetable or synthetic origin, such as peanut oil, soybean oil, mineral oil, sesame oil, etc. Water can be used as a carrier when the pharmaceutical composition is administered intravenously. Saline solutions and aqueous dextrose and glycerol solutions can also be employed as liquid carriers, for example, for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers, such as triglycerides. Oral formulation can include standard carriers, such as pharmaceutical grades of mannitol, lactose, starch, magnesium stearate, sodium saccharine, cellulose, magnesium carbonate, etc. Examples of suitable pharmaceutical carriers are described in REMINGTON'S PHARMACEUTICAL SCIENCES by E. W. Martin. Such compositions will contain a therapeutically effective amount of the compound, often in purified form, together with a suitable amount of carrier so as to provide the form for proper administration to the patient. The formulation should suit the mode of administration.

In one embodiment of the invention, the composition is formulated in accordance with routine procedures as a pharmaceutical composition adapted for intravenous administration to human beings. Typically, compositions for intravenous administration are solutions in sterile isotonic aqueous buffer. Where suitable, the composition may also include a solubilizing agent and a local anesthetic, such as lignocaine, to ease pain at the site of the injection. Generally, the ingredients are supplied either separately or mixed together in unit dosage form, for example, as a dry lyophilized powder, or water-free concentrate, in a hermetically sealed container, such as an ampoule or sachette, indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Accordingly, bendamustine may be used in the manufacture of a medicament. Pharmaceutical compositions of the invention may be formulated as solutions or as lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such a formulation is especially suitable for parenteral administration, but may also be used for oral administration or contained in a metered dose inhaler or nebulizer for insufflation. It may be desirable to add excipients, such as polyvinylpyrrolidone, gelatin, hydroxy cellulose, acacia, polyethylene glycol, mannitol, sodium chloride, or sodium citrate, to such pharmaceutical compositions.

Alternately, bendamustine may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar, or gelatin. Liquid carriers include syrup, peanut oil, olive oil, saline, and water. The carrier may also include a sustained release material, such as glyceryl monostearate or glyceryl distearate, alone or with a wax. The amount of solid carrier varies but, will be between about 20 mg to about 1 g per dosage unit. The pharmaceutical preparations are made following the conventional techniques of pharmacy involving milling, mixing, granulating, and compressing, when suitable, for tablet forms; or milling, mixing and filling for hard gelatin capsule forms. When a liquid carrier is used, the preparation will be in the form of a syrup, elixir, emulsion, or an aqueous, or non-aqueous suspension. Such a liquid formulation may be administered directly by mouth (p.o.) or filled into a soft gelatin capsule.

Bendamustine may be prepared as pharmaceutical compositions containing an effective amount the compound as an active ingredient in a pharmaceutically acceptable carrier. In the compositions of the invention, an aqueous suspension or solution containing bendamustine, buffered at physiological pH, in a form ready for injection may be employed. The compositions for parenteral administration will commonly comprise a solution of the bendamustine or a cocktail thereof dissolved in a pharmaceutically acceptable carrier, such as an aqueous carrier. A variety of aqueous carriers may be employed, e.g., 0.4% saline, 0.3% glycine, and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the bendamustine of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of bendamustine for intramuscular injection could be prepared to contain 1 mL sterile buffered water, and between about 1 ng to about 100 mg, e.g., about 50 ng to about 30 mg, or from about 5 mg to about 25 mg, of bendamustine. Similarly, a pharmaceutical composition of bendamustine for intravenous infusion could be made up to contain about 250 mL of sterile Ringer's solution, and about 1 mg to about 30 mg, or from about 5 mg to about 25 mg of bendamustine. Actual methods for preparing parenterally administrable compositions are well known or will be apparent to those skilled in the art and are described in more detail in, for example, REMINGTON'S PHARMACEUTICAL SCIENCE, 15th ed., Mack Publishing Company, Easton, Pa.

Bendamustine when prepared in a pharmaceutical preparation, may be present in unit dose forms. The appropriate therapeutically effective dose can be determined readily by those of skill in the art. Such a dose may, if suitable, be repeated at appropriate time intervals selected as appropriate by a physician during the response period. In addition, in vitro assays may optionally be employed to help identify optimal dosage ranges. The precise dose to be employed in the formulation will also depend upon the route of administration, and the seriousness of the disease or disorder, and should be decided according to the judgment of the practitioner and each patient's circumstances. Effective doses may be extrapolated from dose-response curves derived from in vitro or animal model test systems.

For bendamustine, the dosage administered to a patient is typically 0.1 mg/kg to 100 mg/kg of the patient's body weight. The dosage administered to a patient may be between 0.1 mg/kg and 20 mg/kg of the patient's body weight, or alternatively, 1 mg/kg to 10 mg/kg of the patient's body weight.

The invention also provides a pharmaceutical pack or kit comprising one or more containers filled with one or more of the ingredients of the pharmaceutical compositions of bendamustine. Optionally associated with such container(s) can be a notice in the form prescribed by a governmental agency regulating the manufacture, use or sale of pharmaceuticals or biological products, which notice reflects approval by the agency of manufacture, use or sale for human administration. In another embodiment of the invention, a kit can be provided with the appropriate number of containers required to fulfill the dosage requirements for treatment of a particular indication.

In another embodiment, bendamustine may be delivered in a vesicle, in particular a liposome (see Langer, Science 249:1527-1533 (1990); Treat, et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 317-327; see generally ibid.).

In yet another embodiment, bendamustine can be delivered in a controlled release system. In one embodiment, a pump may be used (see Langer, supra; Sefton, CRC Crit. Ref Biomed. Eng. 14:201 (1987); Buchwald, et al., Surgery 88:507 (1980); Saudek, et al., N. Engl. J. Med. 321:574 (1989)). In another embodiment, polymeric materials can be used (see MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Ranger, et al., J., Macromol. Sci. Rev. Macromol. Chem. 23:61 (1983); see also Levy, et al., Science 228:190 (1985); During, et al., Ann. Neurol. 25:351 (1989); Howard, et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in MEDICAL APPLICATIONS OF CONTROLLED RELEASE, supra, vol. 2, pp. 115-138 (1984)). Other controlled release systems are discussed in the review by Langer (Science 249:1527-1533 (1990)).

Bendamustine may be administered by any appropriate internal route, and may be repeated as needed, e.g., as frequently as one to three times daily for between 1 day to about three weeks to once per week or once biweekly. Alternatively, bendamustine may be altered to reduce charge density and thus allow oral bioavailability. The dose and duration of treatment relates to the relative duration of the molecules of the present invention in the human circulation, and can be adjusted by one of skill in the art, depending upon the condition being treated and the general health of the patient.

Various delivery systems are known and can be used to administer bendamustine, e.g., encapsulation in liposomes, microparticles, microcapsules, recombinant cells capable of expressing the compound, receptor-mediated endocytosis (see, e.g., Wu, et al., J. Biol. Chem. 262:4429-4432 (1987)), construction of a nucleic acid as part of a retroviral or other vector, etc. Methods of introduction include, but are not limited to, intradermal, intramuscular, intraperitoneal, intravenous, subcutaneous, intranasal, epidural, and oral routes. Bendamustine may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

### Anti-CD20 Antibodies

A physician or veterinarian having ordinary skill in the art can readily determine and prescribe the effective amount of the pharmaceutical composition comprising anti-CD20 antibody. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound which is the lowest dose effective to produce a therapeutic effect. It is preferred that administration be intravenous, intramuscular, intraperitoneal, or subcutaneous. If desired, the effective daily dose of a therapeutic composition may be administered as two, three, four, five, six or more sub-doses administered separately at appropriate intervals throughout the day, optionally, in unit dosage forms. While it is possible for anti-CD20 antibody to be administered alone, it is preferable to administer the compound as a pharmaceutical formulation (composition).

In one embodiment, the human monoclonal antibodies according to the invention may be administered by infusion in a weekly dosage of 10 to 2000 mg/m², normally 10 to 500 mg/m², such as 200 to 400 mg/m², such as 375 mg/m². Such administration may be repeated, e.g., 1 to 8 times, such as 3 to 5 times. The administration may be performed by continuous infusion over a period of from 2 to 24 hours, such as of from 2 to 12 hours.

In another embodiment, the antibodies are administered by slow continuous infusion over a long period, such as more than 24 hours, in order to reduce toxic side effects.

In still another embodiment the antibodies are administered in a weekly dosage of from 250 mg to 2000 mg, such as for example 300 mg, 500 mg, 700 mg, 1000 mg, 1500 mg or 2000 mg, for up to 8 times, such as from 4 to 6 times. The administration may be performed by continuous infusion over a period of from 2 to 24 hours, such as of from 2 to 12 hours. Such regimen may be repeated one or more times as necessary, for example, after 6 months or 12 months. The dosage can be determined or adjusted by measuring the amount of circulating anti-CD20 antibodies upon administration in a biological sample by using anti-idiotypic antibodies which target the anti-CD20 antibodies.

In yet another embodiment, the antibodies are administered by maintenance therapy, such as, e.g., once a week for a period of 6 months or more.

In one embodiment, the present invention provides a pharmaceutical composition comprising a therapeutically effective amount of an anti-CD20 antibody. The pharmaceutical compositions may be formulated with pharmaceutically acceptable carriers or diluents as well as any other known adjuvants and excipients in accordance with conventional techniques, such as those disclosed in Remington: The Science and Practice of Pharmacy, 19th Edition, Gennaro, Ed., Mack Publishing Co., Easton, Pa., 1995. A pharmaceutical composition may include diluents, fillers, salts, buffers, detergents (e.g., a nonionic detergent, such as Tween-80), stabilizers, stabilizers (e.g., sugars or protein-free amino acids), preservatives, tissue fixatives, solubilizers, and/or other materials suitable for inclusion in a pharmaceutical composition. The actual dosage levels of the active ingredients in the pharmaceutical compositions may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

An anti-CD20 antibody of the present invention may be administered via any suitable route, such as an oral, nasal, inhalable, intrabronchial, intraalveolar, topical (including buccal, transdermal and sublingual), rectal, vaginal and/or parenteral route In one embodiment, a pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In one embodiment an anti-CD20 antibody pharmaceutical composition is administered by intravenous or subcutaneous injection or infusion. For example the pharmaceutical composition may be administered over 2-8 hours, such as 4 hours, in order to reduce side effects.

In one embodiment an anti-CD antibody pharmaceutical composition is administered by inhalation. Fab fragments of an anti-CD20 antibodies may be suitable for such administration route, cf. Crowe et al. (Feb. 15, 1994) Proc Natl Acad Sci USA, 91(4):1386-1390.

In one embodiment an anti-CD20 antibody pharmaceutical composition is administered in crystalline form by subcutaneous injection, cf. Yang et al., PNAS USA 100(12), 6934-6939 (2003).

Pharmaceutically acceptable carriers include any and all suitable solvents, dispersion media, coatings, antibacterial and antifungal agents, isotonicity agents, antioxidants and absorption delaying agents, and the like that are physiologically compatible with a compound of the present invention.

Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

Proper fluidity may be maintained, for example, by the use of coating materials, such as lecithin, by the maintenance of the required particle size in the case of dispersions, and by the use of surfactants.

Pharmaceutical compositions containing an anti-CD20 antibody may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Pharmaceutical compositions containing an anti-CD20 antibody may also comprise isotonicity agents, such as sugars, polyalcohols such as mannitol, sorbitol, glycerol or sodium chloride in the compositions.

Pharmaceutically acceptable diluents include saline and aqueous buffer solutions.

The pharmaceutical compositions containing an anti-CD20 antibody may also contain one or more adjuvants appropriate for the chosen route of administration, such as preservatives, wetting agents, emulsifying agents, dispersing agents, preservatives or buffers, which may enhance the shelf life or effectiveness of the pharmaceutical composition. An anti-CD20 antibody the present invention may for instance be admixed with lactose, sucrose, powders (e.g., starch powder), cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol. Other examples of adjuvants are QS21, GM-CSF, SRL-172, histamine dihydrochloride, thymocartin, Tio-TEPA, monophosphoryl-lipid A/microbacteria compositions, alum, incomplete Freund's adjuvant, montanide ISA, ribi adjuvant system, TiterMax adjuvant, Syntex adjuvant formulations, immune-stimulating complexes (ISCOMs), gerbu adjuvant, CpG oligodeoxynucleotides, lipopolysaccharide, and polyinosinic:polycytidylic acid.

Prevention of presence of microorganisms may be ensured both by sterilization procedures and by the inclusion of various antibacterial and antifungal agents, for example, paraben, chlorobutanol, phenol, sorbic acid, and the like. In addition, prolonged absorption of the injectable pharmaceutical form may be brought about by the inclusion of agents which delay absorption, such as aluminum monostearate and gelatin.

The pharmaceutical compositions containing an anti-CD20 antibody may be in a variety of suitable forms. Such forms include, for example, liquid, semi-solid and solid dosage forms, such as liquid solutions (e.g., injectable and infusible solutions), dispersions or suspensions, emulsions, microemulsions, gels, creams, granules, powders, tablets, pills, powders, liposomes, dendrimers and other nanoparticles (see for instance Baek et al., Methods Enzymol. 362, 240-9 (2003), Nigavekar et al., Pharm Res. 21(3), 476-83 (2004), microparticles, and suppositories.

The optimal form depends on the mode of administration chosen and the nature of the composition. Formulations may include, for instance, powders, pastes, ointments, jellies, waxes, oils, lipids, lipid (cationic or anionic) containing vesicles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing may be appropriate in treatments and therapies in accordance with the present invention, provided that the anti-CD20 antibody in the pharmaceutical composition is not inactivated by the formulation and the formulation is physiologically compatible and tolerable with the route of administration. See also for instance Powell et al., “Compendium of excipients for parenteral formulations” PDA J Pharm Sci Technol. 52, 238-311 (1998) and the citations therein for additional information related to excipients and carriers well known to pharmaceutical chemists.

An anti-CD20 antibody may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers, such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art. See e.g., Sustained and Controlled Release Drug Delivery Systems, J. R. Robinson, ed., Marcel Dekker, Inc., New York, 1978.

To administer the pharmaceutical compositions containing an anti-CD20 antibody by certain routes of administration according to the invention, it may be necessary to coat the anti-CD20 antibody with, or co-administer the antibody with, a material to prevent its inactivation. For example, the anti-CD20 antibody may be administered to a subject in an appropriate carrier, for example, liposomes, or a diluent. Liposomes include water-in-oil-in-water CGF emulsions as well as conventional liposomes (Strejan et al., J. Neuroimmunol. 7, 27 (1984)).

Depending on the route of administration, an anti-CD20 antibody may be coated in a material to protect the antibody from the action of acids and other natural conditions that may inactivate the compound. For example, the anti-CD20 antibody may be administered to a subject in an appropriate carrier, for example, liposomes. Liposomes include water-in-oil-in-water CGF emulsions as well as conventional liposomes (Strejan et al., J. Neuroimmunol. 7, 27 (1984)).

Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated. Supplementary active compounds may also be incorporated into the compositions.

Pharmaceutical compositions for injection must typically be sterile and stable under the conditions of manufacture and storage. The composition may be formulated as a solution, microemulsion, liposome, or other ordered structure suitable to high drug concentration. The carrier may be a aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating, such as lecithin, by the maintenance of the required particle size in the case of dispersion and by the use of surfactants. In many cases, it will be preferable to include isotonic agents, for example, sugars, polyalcohols, such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration.

Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

The present invention may be embodied in other specific forms, without departing from the spirit or essential attributes thereof, and, accordingly, reference should be made to the appended claims, rather than to the foregoing specification or following examples, as indicating the scope of the invention.

As used herein, the term, “carrier”, refers to a diluent, adjuvant, excipient, or vehicle with which the therapeutic is administered.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from at least one of its coexisting cellular materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

As used herein, the term, “pharmaceutical”, includes veterinary applications of the invention. The term, “therapeutically effective amount”, refers to that amount of therapeutic agent, which is useful for alleviating a selected condition.

As used herein, the term, “pharmaceutically acceptable”, means approved by a regulatory agency of the Federal or a state government or listed in the U.S. Pharmacopeia or other generally recognized pharmacopeia for use in animals, and more particularly in humans.

For avoidance of doubt, in one embodiment of administering bendamustine with an anti-CD20 antibody is a staggered administration, whereby bendamustine and anti-CD20 antibody is given on alternating basis. For avoidance of doubt, either bendamustine or an anti-CD20 antibody may be administered first for in a staggered administration.

| | | |
|---|---|---|
| SEQ ID | 2F2 V_{H} | EVQLVESGGGLVQPGRSLR |
| NO: 1 | | LSCAASGFTFNDYAMHWV |
| | | RQAPGKGLEWVSTISWNSG |
| | | SIGYADSVKGRFTISRDNA |
| | | KKSLYLQMNSLRAEDTAL |
| | | YYCAKDIQYGNYYYGMDV |
| | | WGQGTTVTVSS |
| SEQ ID | 2F2 V_{L} | EIVLTQSPATLSLSPGERAT |
| NO: 2 | | LSCRASQSVSSYLAWYQQ |
| | | KPGQAPRLLIYDASNRATGI |
| | | PARFSGSGSGTDFTLTISSLE |
| | | PEDFAVYYCQQRSNWPITF |
| | | GQGTRLEIK |
| SEQ ID | 2F2 V_{H} | DYAMH |
| NO: 3 | CDR1 | |
| SEQ ID | 2F2 V_{H} | TISWNSGSIGYADSVKG |
| NO: 4 | CDR2 | |
| SEQ ID | 2F2 V_{H} | DIQYGNYYYGMDV |
| NO: 5 | CDR3 | |
| SEQ ID | 2F2 V_{L} | RASQSVSSYLA |
| NO: 6 | CDR1 | |
| SEQ ID | 2F2 V_{L} | DASNRAT |
| NO: 7 | CDR2 | |
| SEQ ID | 2F2 V_{L} | QQRSNWPIT |
| NO: 8 | CDR3 | |
| SEQ ID | 11B8 V_{H} | DYYGAGSFYDGLYGMDV |
| NO: 9 | CDR3 | |
| SEQ ID | 2F2 V_{H} | DYAMHWVRQAPGKGLEW |
| NO: 10 | CDR1-CDR3 | VSTISWNSGSIGYADSVKG |
| | | RFTISRDNAKKSLYLQMNS |
| | | LRAEDTALYYCAKDIQYG |
| | | NYYYGMDV |
| SEQ ID | 2C6 V_{H} | DNQYGSGSTYGLGV |
| NO: 11 | CDR3 | |

### Example 1

### Non-Limiting Example of Oftatumumab/Bendamustine Combination Administration

In order to treat follicular lymphoma which is refractory to rituximab, in one embodiment, ofatumumab is administered i.v. day 1: 300 mg, day 8: 1000 mg in cycle 1, followed by 1000 mg on day 1 of cycles 2 through 6; and bendamustine is given 60-120 mg/m² in cycles 1 through 6 on days 1 and 2 every 28 days (each cycle is every 28 days);.

In another embodiment, ofatumumab is administered i.v. day 1: 300 mg, day 8: 1000 mg in cycle 1, followed by 1000 mg on day 1 of cycles 2 through 6 (each cycle is every 28 days for ofatumuamb); and bendamustine is given 60-120 mg/m² in cycles 1 through 8 on days 1 and 2 every 21 days (each cycle is every 21 days for bendamustine).

In another embodiment, ofatumumab is administered i.v. day 1: 300 mg, day 8: 1000 mg in cycle 1, followed by 1000 mg on day 1 of cycles 2 through 6; and bendamustine is given 90 mg/m² in cycles 1 through 6 on days 1 and 2 every 28 days (each cycle is every 28 days);.

In another embodiment, ofatumumab is administered i.v. day 1: 300 mg, day 8: 1000 mg in cycle 1, followed by 1000 mg on day 1 of cycles 2 through 6 (each cycle is every 28 days for ofatumuamb); and bendamustine is given 120 mg/m² in cycles 1 through 8 on days 1 and 2 every 21 days (each cycle is every 21 days for bendamustine).

In further embodiment, ofatumumab may be further administered 1000 mg every 2 months for 2 years after the completion of the 6 cycles of ofatumumab (each cycle is every 28 days).

In further embodiment, ofatumumab may be further administered 2000 mg every 2 months after completion of the 6 cycles of ofatumumab (each cycle is every 28 days).

In further embodiment, ofatumumab may be further administered 500 mg every 2 months after completion of the 6 cycles of ofatumumab (each cycles is 28 days).

In further embodiment, ofatumumab may be further administered 500 mg, 1000 mg or 2000 mg every month or every three months after completion of the 6 cycles of ofatumumab (each cycles is 28 days).

In further embodiment, ofatumumab is further administered 300-2000 mg every 2 months for 2 years after the completion the 6 cycles of ofatumumab (each cycles is 28 days).

In further embodiment, ofatumumab may be further administered 300-2000 mg every 2 months for 2 years after the completion of the 6 cycles of ofatumumab (each cycles is 28 days) to those subjects achieving a complete remission (CR), partial remission (PR), or stable disease (SD).

### Example 2

### In Vivo Study Demonstrating Efficacy in Treating Ofatumumab and Bendamustine in CLL Model

Since Rituxan and ofatumumab are anti-human antibodies they need to be directly labeled with a fluorescent tag using a Zenon labeling kit from Invitrogen (Z-25455). One microgram of each antibody was prepared in PBS and five microliters of the Zenon human IgG labeling reagent (Component A) was added to the antibody solution. The mixture was incubated for five minutes at room temperature and then five microliters of the Zenon blocking reagent (Component B) was added to the reaction mixture. After another five minutes at room temperature the complexes were ready to be used. 5×10⁶ cells/ml of viable JVM-3 cells were resuspended in PBS. 100 ul of the cells were added to each tube. 10 ul of human IgG was added to block non-specific binding. The cells and human IgG were incubated for 10 minutes. 10 ul of each fluorescently labeled anti-CD20 antibody was added to the appropriate tube (Rituxan, Ofatumumab and BD Bioscience anti-CD20 antibody clone 2H7). The mixture was incubated for an additional 30 minutes on ice in the dark. Then 500 ul of PBS was added to the cells and they were centrifuged for 5 minutes at 1000 rpm. The supernatant was removed and 500 ul of PBS was added and the cells were centrifuged again. Again the supernatant was removed and the cells resuspended in 300 ul of PBS. The cells were analyzed on a BD FACSCanto.

### Conclusion:

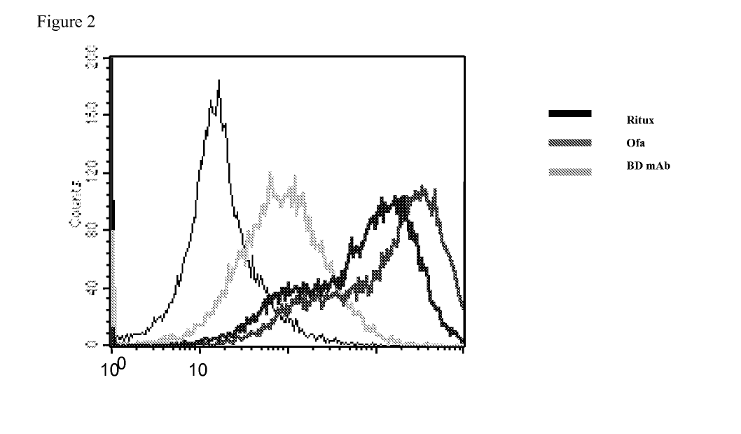

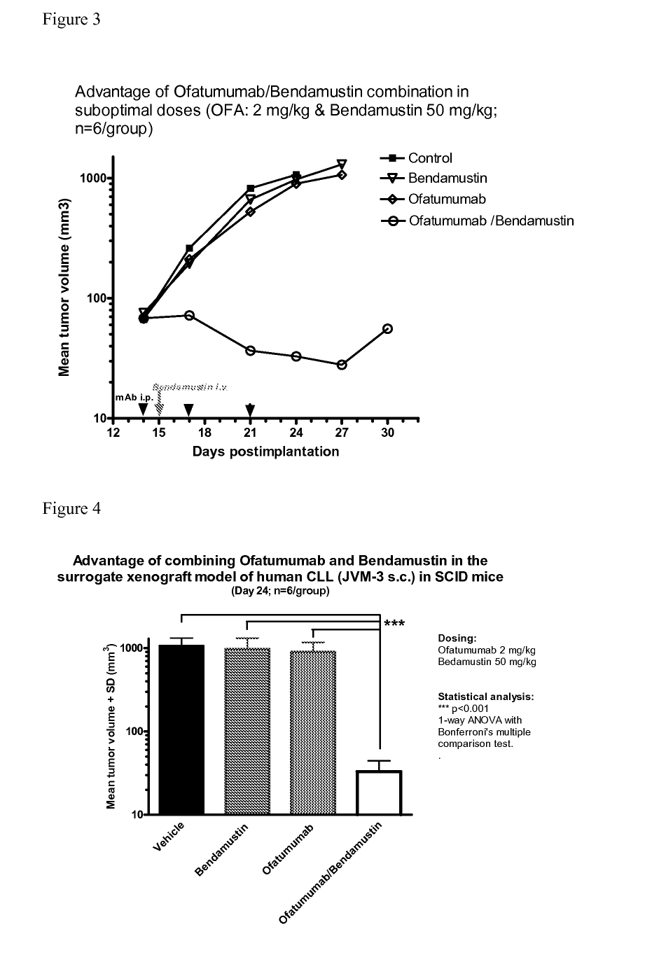

The directly labeled anti-CD20 antibody from BD Bioscience bound less of the receptor on the cell surface than either the rituxan or ofatumumab antibody. This could be due to difference in labeling procedures. However, the Rituxan and ofatumumab were labeled in the same manner and ofatumumab bound more receptors on the cell surface than rituxan antibody did. See FIG. 2.

Human B cell leukemia JVM-3 cell line was obtained from DSMZ (German Collection of Microorganisms and Cell Culture) through a material transfer agreement, and cryopreserved. JVM-3 cells were obtained from the repository and cultured in RPMI 1640 media supplemented with 10% fetal bovine serum, 1% Sodium Pyruvate and 1% Glutamine at 37° C. in a humidified, 5% CO₂ incubator. CB.17-SCID female mice received subcutaneous injections of 4×10⁶ JVM-3 cells in the flank. Tumor diameters were measured twice a week with calipers, and tumor volumes were calculated using formula: volume=width²×length/2. Mice were randomized into therapeutic groups and therapy was initiated on day 14 post-implantation when tumors reached mean volume 66-76 mm³. Treatment groups received ofatumumab 2 mg/kg i.p. twice a week (on days 14, 17 and 21), and/or one injection of alkylating agent bendamustin 50 mg/kg i.v. on day 15. Tumor volume data were graphed using Prism GraphPad software and statistically evaluated with one-way ANOVA followed by Bonferroni multiple comparison test.

### CONCLUSION

Our data demonstrate that combining ofatumumab (2 mg/kg i.p. twice a week) with bendamustin chemotherapy (50 mg/kg i.v. single dose) results in a significant delay of tumor growth as compared to the groups treated with monotherapy (either antibody or bendamustine) or vehicle. Our data shows the benefits of ofatumumab/bendamustin combination therapy in the clinical setting with increased survival and reduced toxicity in CLL patients.

1. A method of treating or preventing a cancer in a patient, comprising the step of administering to the patient bendamustine and an anti-CD20 antibody.

2. The method as claimed in claim 1, wherein the cancer is a lymphoma.

3. The method as claimed in claim 1, in which the cancer is a tumor type which expresses CD20 selected from the group of a precursor B- or T-cell neoplasm, a mature B-cell neoplasm, Hodgkin's lymphoma, or an immunodeficiency associated lymphoproliferative disorder.

4. The method of claim 1 wherein the cancer is rituximab-refractory indolent non-Hodgkin's lymphoma.

5. The method of claim 4 in which the indolent non-Hodgkin's lymphoma is follicular lymphoma.

6. The method as claimed in claim 1 wherein the cancer is selected from the group consisting of NHL (non-Hodgkin's lymphoma), B cell lymphoblastic leukemia/lymphoma, mature B cell neoplasms, B cell chronic lymhocytic leukemia (CLL), small lymphocytic lymphoma (SLL), B cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, anaplastic large-cell lymphoma (ALCL), T-cell Non-Hodgkin's lymphoma; and melanoma.

7. The method as claimed in claim 3 wherein the administration of bendamustine and the anti-CD20 is simultaneous.

8. The method as claimed in claim 1 wherein the administration of bendamustine and the anti-CD20 antibody is sequential, wherein bendamustine is administered first.

9. The method as claimed in claim 1 wherein the administration of bendamustine and the anti-CD20 antibody is sequential, wherein the antibody is administered first.

10. The method as claimed in claim 1 wherein the administration of bendamustine and the anti-CD20 antibody is staggered.

11. The method of claim 3 in which the anti-CD20 antibody is ofatumumab.

12. The method of claim 11 in which ofatumumab is administered i.v. day 1: 300 mg, day 8: 1000 mg in cycle 1, followed by 1000 mg on day 1 of cycles 2 through 6; and bendamustine is given 90 mg/m² in cycles 1 through 6 on days 1 and 2 every 28 days (each cycle is every 28 days).

13. The method of claim 12 in which ofatumumab is further administered 1000 mg every 2 months for 2 years after the completion of the 6 cycles.

14. The method of claim 12 in which ofatumumab is further administered 2000 mg every 2 months after completion of the 6 cycles.

15. The method of claim 12 in which ofatumumab is further administered 500 mg every 2 months after completion of the 6 cycles.

16. The method of claim 12 in which ofatumumab is further administered 500 mg, 1000 mg or 2000 mg every month or every three months after completion of the 6 cycles.

17. The method of claim 12 in which ofatumumab is further administered 300-2000 mg every 2 months for 2 years after the completion the 6 cycles.

18. The method of claim 3 in which ofatumuamb and bendamustine are administered iv.

19. A pharmaceutical composition comprising bendamustine and an anti-CD20 antibody wherein the combination is suitable for separate, sequential and/or simultaneous administration.

20. The pharmaceutical composition according to claim 19, wherein the anti-CD20 antibody is ofatumumab.

21-24. (canceled)
